(19) United States
(12) Patent Application Publication
(10) Pub. No.: US 2007/0016106 A1
(43) Pub. Date: Jan. 18, 2007

(54) Single puncture antegrade-retrograde endovascular sheath

(76) Inventors: Venkatesh Ramaiah, Scottsdale, AZ (US); Robert Myron McNutt, Mesa, AZ (US)

Correspondence Address: SQUIRE SANDERS & DEMPSEY LLP, TWO RENAISSANCE SQUARE, 40 NORTH CENTRAL AVENUE, SUITE 2700, PHOENIX, AZ, 85004-4498, US

(21) Appl. No.: 11/481,575

(22) Filed: Jul. 5, 2006

Related U.S. Application Data
(60) Provisional application No. 60/595,427, filed on Jul. 5, 2005.

(51) Int. Cl. A61M 25/00

(52) U.S. Cl. 600/585

(57) ABSTRACT

The present invention provides a system for directing a first guidewire in a retrograde direction in a blood vessel and for directing a second guidewire in an antegrade direction in a blood vessel. The system includes a dual-lumen obturator having a first lumen leading to a first obturator opening and a second lumen leading to a second obturator opening, and a sheath having a first end having a first sheath opening, a second end having a second opening, a sheath lumen extending from the first end to the second end, and a third sheath opening between the first end and the second end. The first, second, and third sheath openings are in communication with the lumen. A first guidewire is directed through the first lumen and through the second sheath opening in a retrograde direction in the blood vessel, while a second guidewire is directed through the second lumen and through the third sheath opening in an antegrade direction in a blood vessel.

### CROSS REFERENCE TO RELATED APPLICATIONS

This application claims the benefit of U.S. Provisional Application No. 60/595,427, filed Jul. 5, 2005, the contents of which are incorporated by reference herein.

### DESCRIPTION OF THE INVENTION

1. Field of the Invention

The present invention relates generally to medical devices, and more particularly to a system for directing guidewires into a blood vessel in both the retrograde and antegrade direction through a single puncture.

2. Background of the Invention

Percutaneous medical procedures performed on blood vessels (e.g., balloon angioplasties, stent grafts, etc.) are increasingly common. In most cases, a guidewire must be placed in the blood vessel in order to guide catheters, sheaths, and other medical devices to the treatment site. Conventionally, guidewire insertion is performed in a retrograde fashion (i.e., upstream of blood flow). As such, guidewire insertion involves making a puncture with a needle through the skin and into the blood vessel at some point downstream of the treatment site. A guidewire is then introduced into the blood vessel through the needle in the retrograde direction. The needle is removed and a sheath is placed over the guidewire into the vessel lumen. This sheath is now a hemostatic conduit to place balloons, stents, and other devices into the vessel lumen.

In some situations a patient may require treatment of lesions proximal and distal to the puncture site. As such, it is often the case that a second treatment site is antegrade (i.e., downstream of blood flow) from the puncture site used for the first treatment. Antegrade insertion cannot typically be performed at puncture sites first used for retrograde insertion. As such, an additional puncture site for the second treatment site is typically used for an antegrade guidewire insertion. Such an antegrade insertion is generally undesirable. A second, antegrade insertion may have to be substantially delayed from the first guidewire insertion because the first puncture for the first retrograde guide may take from 72 hours to one week to fully heal.

### SUMMARY OF THE INVENTION

The present invention provides a system for directing a first guidewire in a retrograde direction in a blood vessel and for directing a second guidewire in an antegrade direction in a blood vessel through a single puncture. The system includes a dual-lumen obturator having a first lumen leading to a first obturator opening and a second lumen leading to a second obturator opening, and a sheath having a first end having a first sheath opening, a second end having a second opening, a sheath lumen extending from the first end to the second end, and a third sheath opening between the first end and the second end. The first, second, and third sheath openings are in communication with the lumen. A first guidewire is directed through the first lumen and through the second sheath opening in a retrograde direction in the blood vessel, while a second guidewire is directed through the second lumen and through the third sheath opening in an antegrade direction in a blood vessel.

A system, obturator, or sheath according to the invention may have any suitable shape, structure or dimension, and may be expanded and contracted in any suitable manner.

According to one embodiment of the invention, the system may include a sheath that further includes a structure downstream of the third sheath opening, the structure for directing the second guidewire in an antegrade direction. Such a structure may be a balloon.

It is to be understood that the descriptions of this invention herein are exemplary and explanatory only and are not restrictive of the invention as claimed.

### BRIEF DESCRIPTION OF THE DRAWINGS

FIG. 1 shows a dual-lumen obturator according to one embodiment of the invention.

FIG. 2 shows a sheath according to one embodiment of the invention.

FIG. 3 shows one embodiment of the invention in operation.

FIG. 4 shows additional obturators according to various aspects of the invention.

FIG. 5 shows a sheath with an occlusion structure according to one embodiment of the invention.

### DESCRIPTION OF THE EMBODIMENTS

Reference will now be made in detail to the present exemplary embodiments of the invention, examples of which are illustrated in the accompanying drawings.

The present invention provides a system for directing a first guidewire in a retrograde direction in a blood vessel and for directing a second guidewire in an antegrade direction in a blood vessel. The system includes a dual-lumen obturator having a first lumen leading to a first obturator opening and a second lumen leading to a second obturator opening, and a sheath having a first end having a first sheath opening, a second end having a second opening, a sheath lumen extending from the first end to the second end, and a third sheath opening between the first end and the second end. The first, second, and third sheath openings are in communication with the lumen. A first guidewire is directed through the first lumen and through the second sheath opening in a retrograde direction in the blood vessel, while a second guidewire is directed through the second lumen and through the third sheath opening in an antegrade direction in a blood vessel.

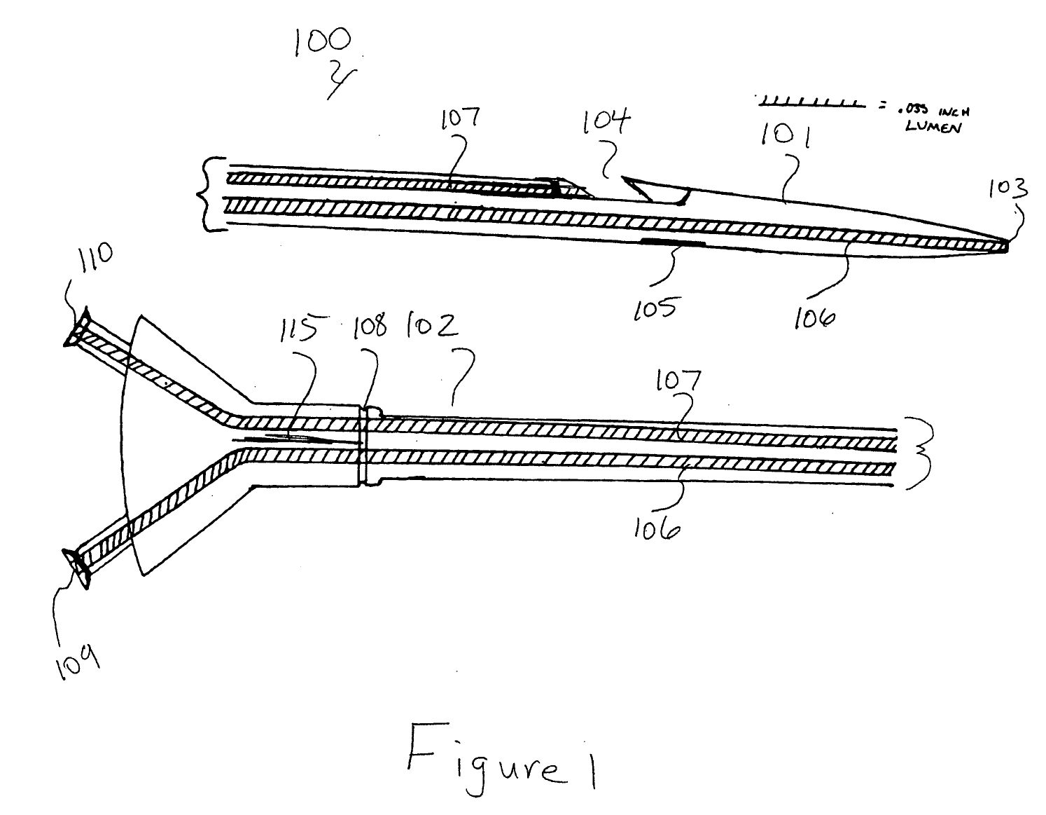

FIG. 1 shows a dual-lumen obturator according to one embodiment of the invention. Obturator 100 has a distal end 101 and a proximal end 102. Distal end 101 is preferably tapered for easier insertion, though a taper is not required. First obturator opening 103 is at the end of distal end 101 and is in fluid communication with first lumen 106. First obturator opening 103 has substantially the same diameter as first lumen 106. Second obturator opening 104 is on the side of obturator 100 and is fluid communication with second lumen 107. Second operator opening 104 is preferably larger in diameter than second lumen 107 so that a guidewire may pass through second operator opening 104 when obturator 100 is bent. In addition, second obturator opening 104 is preferably cut an angle into the side of obturator 100. In this way, the angled second obturator opening 104 allows for antegrade directional influence of a guidewire passed through the second obturator opening. First lumen 106 and second lumen 107 may have any diameter desired for use with different gauge guidewires (e.g., a 0.035 inch guidewire).

Second obturator opening 104 is positioned proximal of first obturator opening 103. A radiopaque marker 105 may be positioned on obturator 100 opposite second obturator opening 104 in order to aid an operator in detecting and positioning second obturator opening 104 of obturator 100.

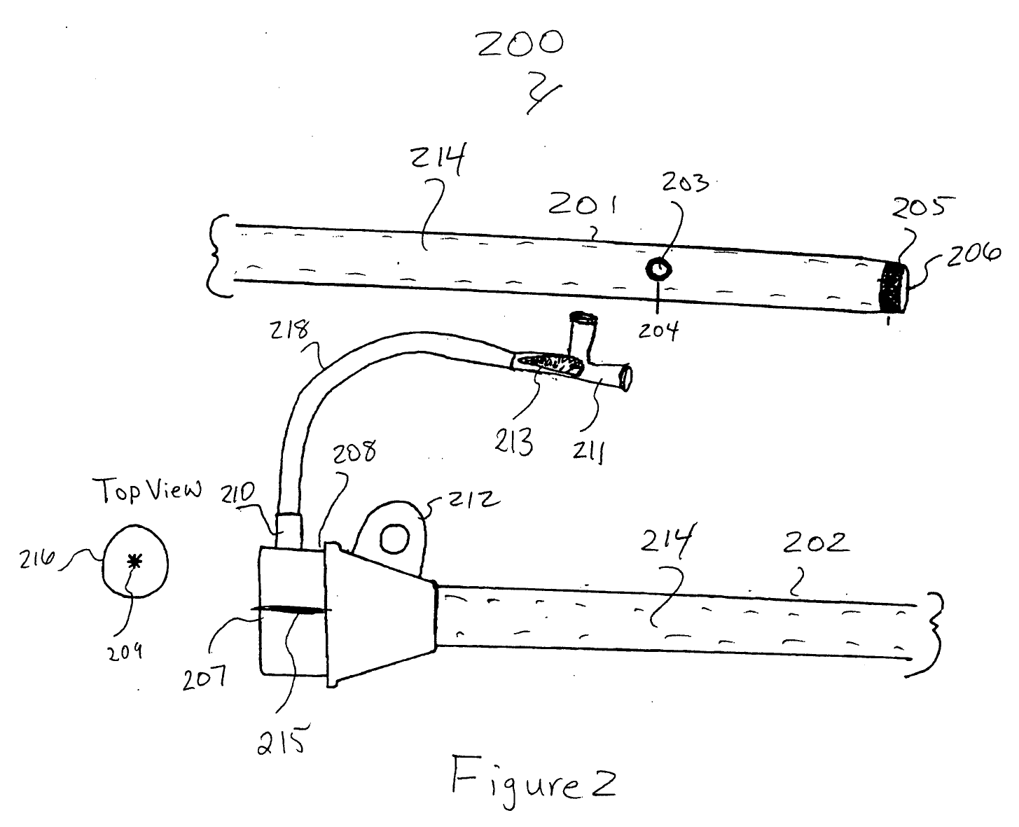

Proximal end 102 includes a locking hub 108 which locks obturator 100 into hemostatic valve 208 of sheath 200 (see FIG. 2). As shown in FIG. 1, proximal end 102 may also be flared to prevent the obturator from being pushed completely into a sheath. Proximal opening 109 is in fluid communication with first lumen 106. Proximal opening 110 is in fluid communication with second lumen 107. Proximal end 102 may also include an alignment mark 115 for aiding a user in aligning the obturator with sheath 200 (see FIG. 2).

As shown in FIG. 1, the dashed lines in the first and second lumens represent 0.035 inch increments. Preferably, obturator 100 is 6 to 9 French in diameter. However, any size (both length and diameter) or shape obturator may be constructed for use in any blood vessel. Obturator 100 is preferably made from a plastic or resin material, but may be made of any material suitable for use within a blood vessel. Preferably, obturator 100 is made from a flexible material so that it is bendable at least at second obturator opening 104.

FIG. 2 shows a sheath according to one embodiment of the invention. Sheath 200 includes a distal end 201 and a proximal end 202. Second sheath opening 206 is in fluid communication with sheath lumen 214. Second sheath opening 206 may include a radiopaque marker 205 to aid a user in detection and placement of the sheath. Second sheath opening 206 is preferably 6 to 9 French in diameter, however, any diameter opening may be used. Likewise, any suitable diameter of sheath may be used for use in any blood vessel. Preferably, second sheath opening 206 is constructed so that it becomes occluded when obturator 100 is placed into the sheath and through second sheath opening 206. Sheath 200 also includes a third sheath opening 203 in fluid communication with sheath lumen 214. Third sheath opening 203 is preferably 3 to 7 French in inner diameter and is 3 to 5 cm upstream from second sheath opening 206. However, third sheath opening 203 may be positioned at any suitable distance away from second sheath opening 206 and may be constructed in any suitable diameter to accommodate varying guidewire sizes. Third sheath opening 203 may include a radiopaque marker 204 to aid a user in the placement and positioning of the sheath. For example, alignment of radiopaque marker 204 with radiopaque marker 105 on obturator 100 (see FIG. 1) indicates to that third sheath opening 203 and second obturator opening 104 are aligned (i.e., in fluid communication with each other).

Proximal end 201 includes a first sheath opening 207 in fluid communication with sheath lumen 214. First sheath opening 207 is covered by hemostatic valve 208. Hemostatic valve 208 may include a top plate 216 with a rubber grommet. Obturators, guidewires, and other devices may be inserted into hemostatic valve 108 through pattern 209 cut into top plate 216. Pattern 209 exposes the slits in the rubber grommet. These slits allow devices to be inserted into the sheath lumen, while preventing blood and other fluids from coming out the proximal end of the sheath. However, any type of hemostatic valve may be used.

Proximal end 201 may further include an injection port 210. Injection port 210 is in fluid communication with sheath lumen 214. Injection port 210 may be further connected to valve mechanism 211 with three-way stopcock 213 through tubing 218. Valve mechanism 211 with three-way stopcock 213 allows for the controlled release of fluids (e.g., saline, medicine, etc.) into sheath 200. Proximal end 201 may further include anchoring appendage 212 for securing sheath 200 to a patient with a suture. Proximal end 201 may also include an alignment mark 215. When obturator 100 is locked into hemostatic valve 208, alignment of alignment mark 215 with alignment mark 115 of obturator 100 indicates to a user that third sheath opening 203 is aligned with second obturator opening 104 (i.e., they are in fluid communication with each other).

Preferably, sheath 200 is approximately 11 to 15 cm in length, however any suitable length may be used. Preferably, sheath 200 is constructed from a flexible plastic material or kink-resistance metal braid, however, any sheath 200 may be constructed from any material suitable for use within a blood vessel. Preferably, sheath 200 is made from a flexible material so that it is bendable at least at third sheath opening 203.

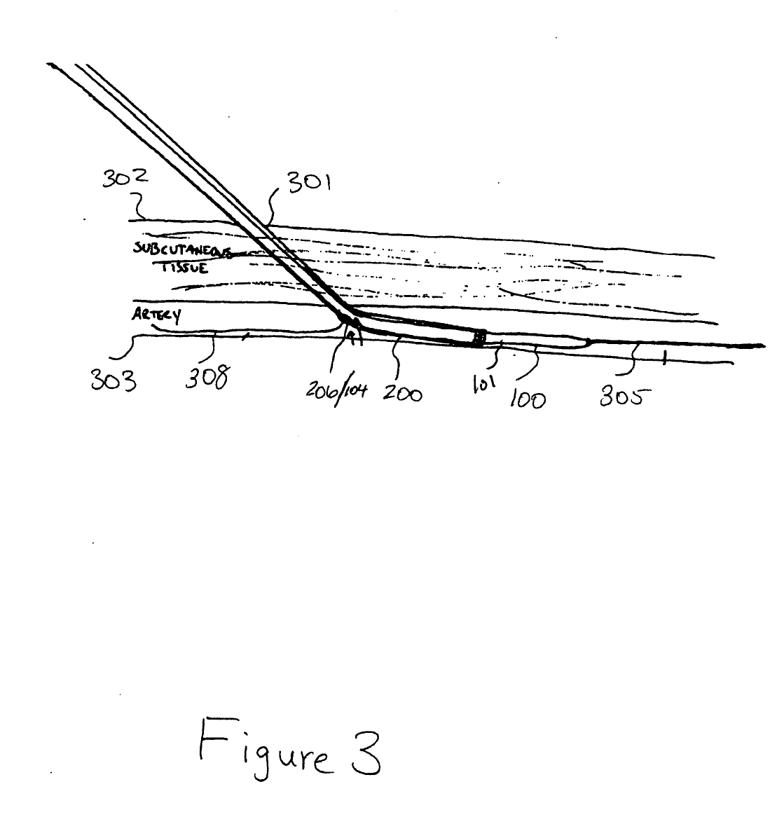

FIG. 3 shows one embodiment of the invention in operation. Initially a puncture 301 is made through subcutaneous tissue 302 and into a blood vessel 303 (e.g., an artery) of a patient. Next an obturator 100 is fully inserted into a sheath 200 and placed through the puncture and into the artery so that distal end 101 of obturator 100 is pointing in the retrograde direction (i.e., going against the flow of blood). Now, a first guidewire 305 may be inserted through the first lumen 106 of obturator 100 and into blood vessel 303 in the retrograde direction.

To place a guidewire in the antegrade direction, alignment marks on obturator 100 and sheath 200 should be aligned to ensure that third sheath opening 206 is aligned with second obturator opening 104. As described above, alignment may be accomplished using radiopaque markers near the respective openings of the obturator or sheath or by using alignment marks on the proximal ends of the obturator and sheath. Once third sheath opening 206 and second obturator opening 104 are aligned, pulsatile blood flow should be seen at proximal opening 110 of obturator 100. If no blood flow is detected, the system (i.e., the obturator 100/sheath 200 combination) should be pushed further into blood vessel 303 until pulsatile blood flow is detected at proximal opening 110. If blood flow is still not detected, alignment of obturator 100 and sheath 200 should be rechecked.

Once proper alignment is established through detection of pulsatile blood flow, the system should be pulled out of blood vessel 303 until pulsatile blood flow is no longer detected at proximal opening 110. Then, the system should be pushed back into the blood vessel just until pulsatile blood flow returns at proximal opening 110. In this way, third sheath opening 206 and second obturator opening 104 are positioned just inside of blood vessel 103. Next, the system should be flexed or bent so that third sheath opening 206 and second obturator opening 104 are pointing in the antegrade direction. Now a second guidewire 308 may be inserted through second lumen 107 of obturator 100 and into blood vessel 303 in the antegrade direction. Then, both obturator 100 and sheath 200 may be removed from the patient, leaving the two guidewires. Sheath 200 or another conventional sheath may then be placed over the antegrade guidewire for antegrade lesion intervention. The retrograde guidewire may be left in place to maintain retrograde access for further retrograde interventions or for vessel closure device insertion.

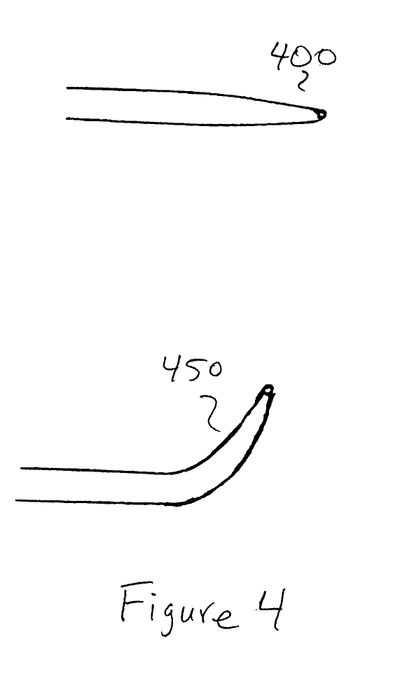

FIG. 4 shows additional obturators according to various aspects of the invention. Obturator 400 is a conventional single lumen obturator. This obturator may be useable with sheath 200 in situations where it is desired to only place a guidewire in a single direction. Obturator 450 is also a single-lumen obturator. However it contains a curved tip in order to aid placement of a sheath in the antegrade direction.

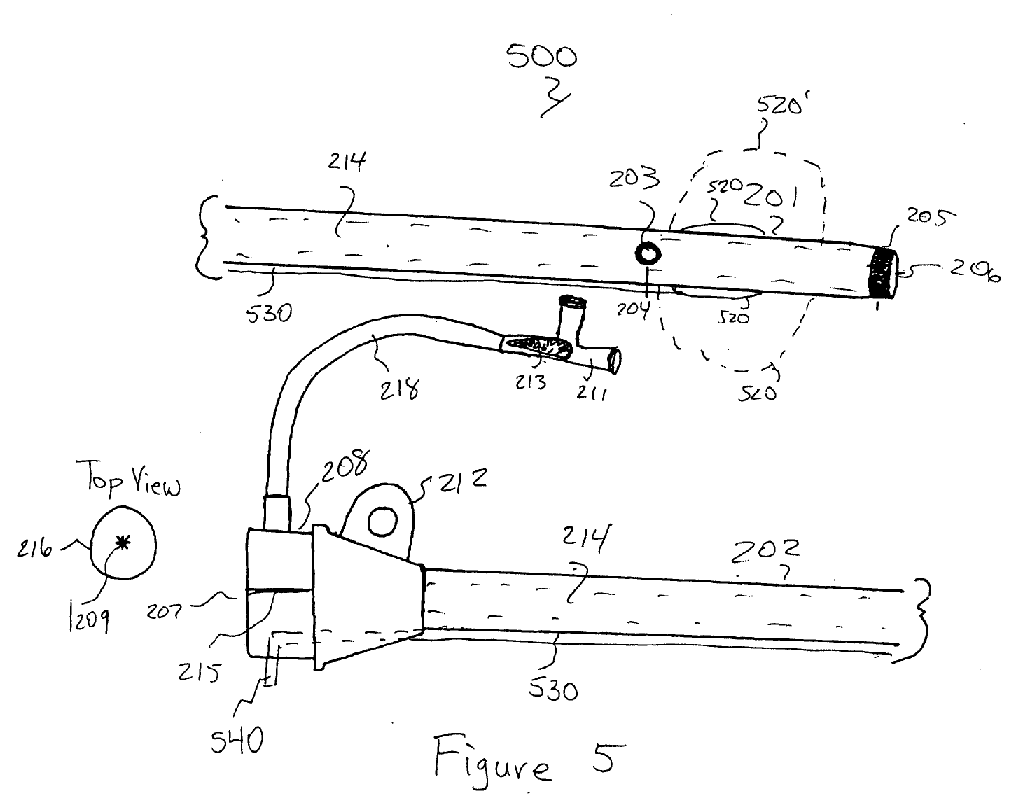

FIG. 5 shows a sheath with an occlusion structure according to one embodiment of the invention. This sheath is substantially the same as sheath 200 shown in FIG. 2. However, sheath 500 includes a structure 520 positioned downstream of third sheath opening 203. Structure 520 is constructed so that it may be inflated or expanded when the sheath/obturator system is in place for antegrade insertion of a guidewire. Element 520′ shows structure 520 in an expanded/inflated state. When inflated or expanded, structure 520 prevents a guidewire exiting third sheath opening 203 from going in the retrograde direction. Instead a guidewire would be deflected off structure 520 and pushed toward the antegrade direction. Preferably, structure 520 is a balloon-like structure. If structure 520 is a balloon-like structure, sheath 500 would further include a tube 530 and an additional injection port 540. Injection port 540 is in fluid communication with structure 520 through tube 530. Tube 530 may be placed on the outside of sheath 500 or may be routed substantially through lumen 214. Structure 520 may be inflated by filling it with liquid (e.g., saline) or gas (e.g., air) provide through injection port 540.

Other embodiments of the invention will be apparent to those skilled in the art from consideration of the specification and embodiments disclosed herein. Thus, the specification and examples are exemplary only, with the true scope and spirit of the invention set forth in the following claims and legal equivalents thereof.

What is claimed is:

1. A system for directing a first guidewire in a retrograde direction in a blood vessel and for directing a second guidewire in an antegrade direction in a blood vessel, the system comprising:
(a) a dual-lumen obturator having a first lumen leading to a first obturator opening and a second lumen leading to a second obturator opening;
(b) a sheath having a first end having a first sheath opening, a second end having a second opening, a sheath lumen extending from the first end to the second end, and a third sheath opening between the first end and the second end, the first, second, and third sheath openings in communication with the lumen;
whereby the first guidewire is directed through the first lumen and through the second sheath opening in a retrograde direction in the blood vessel, and the second guidewire is directed through the second lumen and through the third sheathing opening in an antegrade direction in a blood vessel.

2. The system of claim 1 wherein a radiopaque marker is positioned on or near the second obturator opening and the third sheath opening.

3. The system of claim 1 wherein the sheath further comprises a structure downstream of the third sheath opening, the structure for directing the second guidewire in an antegrade direction.

4. The system of claim 3 wherein the structure is a balloon.

5. A dual-lumen obturator having a first obturator lumen with a first obturator opening and a second obturator lumen with a second obturator opening, the second opening being antegrade to the first opening.

6. A sheath having a first end, a second end, a lumen extending between the first end and the second end and an opening between the first end and the second end, the opening in communication with the lumen.

7. The system of claim 6 wherein the sheath further comprises a structure downstream of the opening, the structure for directing a guidewire in an antegrade direction.

8. The system of claim 7 wherein the structure is a balloon.
